(19) United States
(12) Patent Application Publication
(10) Pub. No.: US 2012/0028338 A1
(43) Pub. Date: Feb. 2, 2012

(54) MIXOTROPHIC ALGAE FOR THE PRODUCTION OF ALGAE BIOFUEL FEEDSTOCK ON WASTEWATER

(76) Inventors: Ashish Bhatnagar, Rajasthan (IN); Senthil Chinnasamy, Tamil Nadu (IN); Keshav C. Das, (US)

(21) Appl. No.: 13/257,351

(22) PCT Filed: Apr. 20, 2010
(86) PCT No.: PCT/US10/31683

§ 371 Date: Sep. 19, 2011

Related U.S. Application Data
(60) Provisional application No. 61/170,683, filed on Apr. 20, 2009.

(51) Int. Cl. C12N 1/12

(52) U.S. Cl. 435/257.3; 435/257.1; 435/257.6; 435/257.5

(57) ABSTRACT

The disclosure encompasses, among other aspects, mixed algal populations able to survive and proliferate on culture media that have a high proportion of an industry wastewater. In particular, at least one strain of an alga in the algal population proliferates mixotrophically. Embodiments further encompass methods of cultivating mixed populations of freshwater and marine alga comprising a plurality of genera and species to provide a biomass from which may be extracted lipids, or converted into biodiesel by such procedures as pyrolysis. Lipid material extracted from the algae may be converted to biodiesel or other organic products. Native algal strains were isolated from industrial and in particular agricultural wastewater inoculated with mixed populations derived from environments exposed to such wastewater. Both fresh water and marine algae showed good growth in wastewaters. About 65% of the algal oil obtained from the algal consortium cultured on an industry wastewater could be converted into biodiesel.

### CROSS-REFERENCE TO RELATED APPLICATIONS

This application claims priority to U.S. Provisional Patent Application Ser. No. 61/170,683, entitled “MIXOTROPHIC ALGAE AND THEIR CONSORTIA FOR THE PRODUCTION OF ALGAE BIOFUEL FEEDSTOCK IN WASTEWATER FED OPEN PONDS” filed on Apr. 20, 2009, the entirety of which is hereby incorporated by reference.

### STATEMENT REGARDING FEDERALLY SPONSORED RESEARCH OR DEVELOPMENT

This invention was made with government support under Grant No.: DE-FG36-05G085012 awarded by the Department of Energy of the United States Government. The government has certain rights in the invention.

### TECHNICAL FIELD

The present disclosure is generally related to mixed algal compositions able to proliferate on industrial wastewater, and to methods of obtaining an algal biomass from such cultures for use in generating a biofuel.

### BACKGROUND

Various estimates support that apart from drinking water, farmers will need about 4000 cubic kilometers of water in 2050, as against the current 2700 cubic kilometers, if no new technological changes are deployed to reduce water usage (Amarsinghe et al., (2007) IWMI Research Report 123). Of the estimated water use, the global target for biofuel feedstock crop production for 2030 itself would demand 180 cubic kilometers of water (IWMI, (2008) Water Policy Brief, Issue 30). Algae are considered an economically viable alternative to present biofuel crops such as corn and soybean as they do not require arable land (Chisti Y. (2007) Biotechnol. Adv. 25: 294-306; Hu et al., (2008) Plant J. 54: 621-639). However, their water demand is as high as 11-13 million liters per hectare for cultivation in open ponds. Their ability to grow in industrial, municipal and agricultural wastewaters and seawater can not only overcome this hurdle, but also can simultaneously provide treated water suitable for other uses. Oswald, as early as 1963 (Dev. Ind. Microbiol. 4: 112-119) honed this process of phycoremediation of wastewaters and suggested a number of byproduct applications for the biomass generated.

Besides agricultural use of water, mainly as irrigation, annual water use for domestic purposes between 1987 and 2003 was estimated as about 325 billion cubic meters. Industries consumed 665 billion cubic meters water annually during the same period. Most wastewater is polluting and creating health hazards. If 50% of this non-agricultural consumed water is available for algae production, it would have the potential to generate up to about 250 million tons of algal biomass, including 37 million tons of oil. However, variations in the compositions of wastewaters limit those algae species that may be useful for cultivation on wasterwater.

For economic viability, the cost of production needs to be significantly lowered. Utilization of wastes and wastewaters to cultivate algae can provide solutions to the problems of freshwater demand, can supply cheap nutrients, and can offer remediation of wastes. However, many of the waste streams that are rich in nutrients are colored or dark material such as, for example, carpet industry effluents, poultry litter extracts, slaughterhouse wastes, dairy effluents, swine wastes, municipal waste and wastewater, compost plant/landfill leachate and biogas plant slurry. Growth, therefore, of many species of algae in these waters is affected adversely by their dependence on photosynthesis. There are a number of algae that are facultatively heterotrophic and prefer, when available, an organic carbon substrate over the fixation of carbon dioxide. Other algae, termed mixotrophic, can simultaneously drive photoautotrophy and heterotrophy to utilize both inorganic (CO₂) and organic carbon substrates. This process leads to an additive or synergistic effect of the two processes that enhances their productivity and in turn provides the ability to grow in such wastewaters.

### SUMMARY

One aspect of the present disclosure encompasses methods of generating an algal biomass, comprising: (a) forming an algal culture by combining: (i) a population of algal cells characterized as proliferating in a culture medium comprising an industry wastewater; (ii) a culture medium comprising an industry wastewater, optionally a municipal sewage effluent, and optionally a nutritional supplement, where said nutritional supplement increases the yield of algal culture compared to when the culture medium does not comprise the nutritional supplement, said nutritional supplement comprising an organic carbon source suitable for supporting the proliferation of a mixotrophic algal species, a mineral, a buffer, or a combination thereof; and (b) maintaining the algal culture under conditions suitable for the proliferation of the population of algal cells, thereby forming an algal biomass.

In embodiments of the methods of this aspect of the disclosure, the effluent can be from a poultry industry, a non-poultry meat industry, a plant-based industry, or from a non-agricultural industry.

In the embodiments of this aspect of the disclosure, the population of algal cells can comprise a freshwater (non-marine) algal strain, a plurality of freshwater (non-marine) algal strains, a plurality of cyanobacter strains, a plurality of diatomaceous algal strains, or any combination thereof, where at least one species of the population of algal cells is a mixotrophic alga.

In some embodiments of this aspect of the disclosure, the population of algal cells can comprise a strain of an algal genus selected from the group consisting of: Scenedesmus, Chlorella, Chlamydomonas, Scenedesmus and Chorella, Scenedesmus and Chlamydomonas, Chorella and Chlamydomonas, and Scenedesmus, Chorella, and Chlamydomonas.

In certain embodiments of this aspect of the disclosure, the population of algal cells can be a consortium of algal cells comprising Chlamydomonas globosa, Chlorella minutissima, and Scenedesmus bijuga, and optionally Chlorella sorokiniana.

Yet another aspect of the disclosure encompasses methods of producing a biofuel from industrial wastewater comprising: (a) forming an algal culture by combining: (i) a population of algal cells characterized as proliferating in a culture medium comprising an industry wastewater; (ii) a culture medium comprising an industry wastewater, optionally a municipal sewage effluent, and optionally a nutritional supplement, where the nutritional supplement increases the yield of algal culture compared to when the culture medium does not comprise the nutritional supplement, the nutritional supplement comprising an organic carbon source suitable for supporting the proliferation of a mixotrophic algal species, a mineral, a buffer, or a combination thereof, and where when the industry wastewater is an agricultural industry effluent, the agricultural industry is a poultry industry, a non-poultry meat industry, or a crop-based industry; (b) maintaining the algal culture under conditions suitable for the proliferation of the population of algal cells, thereby forming an algal biomass; (c) isolating the algal biomass from the medium; and (d) obtaining from the isolated algal biomass a biofuel or a source of a biofuel, where the step of obtaining from the isolated biomass a biofuel comprises the steps of isolating a lipid material from the biomass or converting the biomass to a biofuel, and where the isolated lipid material is converted to a biofuel.

### BRIEF DESCRIPTION OF THE DRAWINGS

Further aspects of the present disclosure will be more readily appreciated upon review of the detailed description of its various embodiments, described below, when taken in conjunction with the accompanying figures.

FIG. 1 is a graph showing the percent change in chlorophyll a and biomass content of mixotrophic and heterotrophic growth of algal strains relative to phototrophic growth. D+G, dark+glucose; L+G, light+glucose; CG, Chlamydomaonas globosa; CM, Chlorella minutissima; SB, Scenedesmus bijuga.

FIG. 2 is a graph showing the reduction in chlorophyll a content under mixotrophic and heterotrophic conditions compared with phototrophy. D+G, dark+glucose; L+G, light+glucose; light only; CG, Chlamydomaonas globosa; CM, Chlorella minutissima; SB, Scenedesmus bijuga.

FIG. 3 is a graph showing the percent change in chlorophyll a in mixotrophic algal strains while using various carbon sources compared to phototrophic growth. CG, Chlamydomonas globosa; CM, Chlorella sorokiniana; CM, Chlorella minutissima; SB, Scenedesmus bijuga. Carbon sources: AL, acetate+light; AD, acetate+dark; GL, glucose+light; GD, glucose+dark; GlyL, glycerol+light; GlyD, glycerol+dark; ML, methanol+light; MD, methanol+dark; SL, sucrose+light; SD, sucrose+dark.

FIGS. 4A-4C show a series of graphs illustrating the performance of algae in poultry litter extract (FIG. 4A), carpet industry treated wastewater (FIG. 4B), and untreated (FIG. 4C) wastewater in terms of percent change in biomass (light bars) and chlorophyll a production (dark bars) with reference to the biomass and chlorophyll a content obtained when grown in BG 11 medium. In each of FIGS. 4A-4C, +N and −N indicates whether nitrogen (250 mg/L) has been added as sodium nitrate. CG, Chlamydomonas globosa; CM, Chlorella minutissima; SB, Scenedesmus bijuga. Bars indicate mean values of triplicates and error bars indicate standard deviation.

FIG. 5 is a graph showing the performance of double and triple combinations of algae in poultry litter extract (pale grey bars) and carpet industry untreated wastewater (dark grey bars) in terms of percent change in biomass production with reference to the biomass obtained in BG 11 medium. In each figure, +N and −N indicates whether nitrogen (250 mg/L) has been added as sodium nitrate. GB, C. globosa+S. bijuga; GM, C. globosa+C. minutissima; BM, S. bijuga+C. minutissima; GMB, C. globosa+C. minutissima+S. bijuga. Bars indicate mean values of triplicates. Error bars indicate standard deviation. Solid bars, untreated carpet industry wastewater; open bars, poultry litter extract.

FIG. 6 shows a bar graph illustrating the growth performance of mixotrophic algal strains in terms of chlorophyll a content in BG11 and deionized water supplemented with 5%, 10%, or 15% lignocellulosic sugar hydrolysates. CSO, Chlorella sorokiniana; CM, Chlorella minutissima; CG, Chlamydomonas globosa; SB, Scenedesmus bijuga. BG5, BG10, and BG15 denote BG11 medium supplemented with 5%, 10%, or 15% hydrolysates, respectively; DI5, DI10, and DI15 denote deionized water supplemented with 5%, 10%, or 15% hydrolysates, respectively.

FIG. 7 shows a bar graph illustrating the growth performance of mixotrophic algal strains in terms of biomass productivity in BG11 and deionized water supplemented with 5%, 10%, or 15% lignocellulosic sugar hydrolysates. CSO, Chlorella sorokiniana; CM, Chlorella minutissima; CG, Chlamydomonas globosa; SB, Scenedesmus bijuga. BG5, BG10, and BG15 denote BG11 medium supplemented with 5%, 10%, or 15% hydrolysates, respectively; DI5, DI10, and DI15 denote deionized water supplemented with 5%, 10%, or 15% hydrolysates, respectively.

FIG. 8 shows a bar graph illustrating the growth performance of mixotrophic algal strains in terms of protein content in BG11 or deionized water supplemented with 5%, 10%, or 15% lignocellulosic sugar hydrolysates. CSO, Chlorella sorokiniana; CM, Chlorella minutissima; CG, Chlamydomonas globosa; SB, Scenedesmus bijuga. BG5, BG10, and BG15 denote BG11 medium supplemented with 5%, 10%, or 15% hydrolysates, respectively; D15, DI10, and D115 denote deionized water supplemented with 5%, 10%, or 15% hydrolysates, respectively.

FIG. 9 shows a bar graph illustrating the growth performance of mixotrophic algal strains in terms of carbohydrates content in BG11 or deionized water supplemented with 5%, 10%, or 15% lignocellulosic sugar hydrolysates. CSO, Chlorella sorokiniana; CM, Chlorella minutissima; CG, Chlamydomonas globosa; SB, Scenedesmus bijuga. BG5, BG10 and BG15, BG11 medium supplemented with 5%, 10%, or 15% hydrolysates, respectively; D15, DI10 and DI15, Deionized water supplemented with 5%, 10%, or 15% hydrolysates, respectively.

FIG. 10 shows a bar graph illustrating sugar utilization by mixotrophic algae in BG 11 medium supplemented with 5% lignocellulosic hydrolysates. CSO, Chlorella sorokiniana; CM, Chlorella minutissima; CG, Chlamydomonas globosa; SB, Scenedesmus bijuga.

FIG. 11 shows a bar graph illustrating sugar utilization by mixotrophic algae in BG 11 medium supplemented with 10% lignocellulosic hydrolysates. CSO, Chlorella sorokiniana; CM, Chlorella minutissima; CG, Chlamydomonas globosa; SB, Scenedesmus bijuga.

FIG. 12A shows a bar graph illustrating changes in percent lipid of Chlorella sorokiniana grown in growth medium (BG) or deionized water (DI) supplemented with 5%, 10%, or 15% of lignocellulosic hydrolysates.

FIG. 12B shows a bar graph illustrating changes in percent lipid of Chlorella minutissima grown in growth medium (BG) or deionized water (DI) supplemented with 5%, 10%, or 15% of lignocellulosic hydrolysates.

The details of some exemplary embodiments of the methods and systems of the present disclosure are set forth in the description below. Other features, objects, and advantages of the disclosure will be apparent to one of skill in the art upon examination of the following description, drawings, examples and embodiments. It is intended that all such additional systems, methods, features, and advantages be included within this description, be within the scope of the present disclosure.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a support” includes a plurality of supports. In this specification and in the embodiments that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### DEFINITIONS

In describing the disclosed subject matter, the following terminology will be used in accordance with the definitions set forth below.

The terms “wastewater” and “wastewater effluent” as used herein refer to any discharge from an industrial plant, an industrial process, or an agricultural facility and which may support the growth thereon of an algal population. It is contemplated that an agricultural wastewater may include, but is not limited to, the waste discharge from an animal rearing or growing facility such as a poultry farm, a cattle farm, a sheep farm, a pig farm, and the like. Such waste discharge may include the urine and fecal matter from the animals as well as food residues. Agricultural waste may also include waste discharge from a crop farm, including water used to wash or process vegetable crops, fertilizer or irrigation run-off, and the like. Accordingly, agricultural wastewater can be a rich source of nutrients or diluted to allow treatment in a wasterwater treatment facility using such processes as activated sludge treatment.

Non-agricultural wastewater may be, but is not limited to, a discharge from a manufacturing facility and which may include wastewater from the preparation of raw materials used in a manufacturing process, or from the process itself. Typically, such wastewater comprises the chemical components resulting from the preparation of materials including, but not limited to, organic substances, raw materials thereof, metal ions, acids, alkalis, salts, dye components and the like. Wastewater for use as an algal growth medium as understood in the present disclosure may also be an aqueous extract of a solid waste material such as, but not limited to, an agricultural waste such as a poultry litter. This material may include, but is not limited to, the floor coverings of poultry rearing houses that has been soiled with the waste material of the animals. The solid or semi-solid material with a significant organic carbon, nitron and phosphorous content may be mixed with water for a period to dissolve some or all of the components thereof, filtered to remove residual material and used as a culture medium or to supplement (enrich) another composition comprising the algal growth medium.

The term “untreated wastewater” as used herein refers to water effluent directly from a carpet manufacturing plant without removal of any materials used in, or resulting from, the manufacturing process. The “untreated wastewater” may then be supplemented with effluent from a municipal sewage system that includes in varying amounts residential and commercial sewage.

The term “treated wastewater” as used herein refers to effluent wastewater from a carpet manufacturing facility that has been combined with an amount of a municipal (residential and commercial) sewage and which has then been processed in a sewage or water treatment plant such as by an activated sludge process for the removal or reduction in the level of the carbon and biological loads, metals, etc. Typically, the treated wastewater can be contained within a reservoir open to the atmosphere before disposal such as by spraying onto to land surfaces for further treatment, and while rendered suitable for adding to general sewage or land disposal may include dye components, organic material and the like that can support the growth of microorganisms, including algae.

The term “mixotroph” as used herein refers to a (micro)organism that can use a mix of different sources of energy and carbon. Possible are alternations between photo- and chemotrophy, between litho- and organotrophy, between auto- and heterotrophy or a combination of it. Mixotrophs can be either eukaryotic (for example only, a Chlorella sp., or other alga) or prokaryotic (a cyanobacter). They can take advantage of different environmental conditions. If a trophic mode is obligate, then it is always necessary for sustaining growth and maintenance; if facultative, it can be used as a supplemental source. Some organisms have incomplete Calvin cycles, so they are incapable of fixing carbon dioxide and must use organic carbon sources.

The terms “alga” and “algae” as used herein refer to any organisms with chlorophyll and, in other than unicellular algae, a thallus not differentiated into roots, stems and leaves, and encompasses prokaryotic and eukaryotic organisms that are photoautotrophic or facultative heterotrophs. The term “algae” includes macroalgae (such as seaweed) and microalgae. For embodiments of the disclosure, algae that are not macroalgae can be advantageous. The terms “microalgae” and “phytoplankton,” used interchangeably herein, refer to any microscopic algae, photoautotrophic or facultative heterotroph protozoa, photoautotrophic or facultative heterotroph prokaryotes, and cyanobacteria (commonly referred to as blue-green algae and formerly classified as Cyanophyceae). The use of the term “algal” also relates to microalgae and thus encompasses the meaning of “microalgal.” The term “algal composition” refers to any composition that comprises algae, and is not limited to the body of water or the culture in which the algae are cultivated. An algal composition can be an algal culture, a concentrated algal culture, or a dewatered mass of algae, and can be in a liquid, semi-solid, or solid form. A non-liquid algal composition can be described in terms of moisture level or percentage weight of the solids. An “algal culture” is an algal composition that comprises live algae.

The algae of the disclosure can be a naturally occurring species, a genetically selected strain, a genetically manipulated strain, a transgenic strain, or a synthetic algae. Algae from tropical, subtropical, temperate, polar or other climatic regions can be used in the disclosure. Endemic or indigenous algal species are generally preferred over introduced species where an open culturing system is used. Algae, including microalgae, inhabit all types of aquatic environment, including but not limited to freshwater (less than about 0.5 parts per thousand (ppt) salts), brackish (about 0.5 to about 31 ppt salts), marine (about 31 to about 38 ppt salts), and briny (greater than about 38 ppt salts) environment. Any of such aquatic environments, freshwater species, marine species, and/or species that thrive in varying and/or intermediate salinities or nutrient levels, can be used in the embodiments of the disclosure. The algae in an algal composition of the disclosure may contain a mixture of prokaryotic and eukaryotic organisms, wherein some of the species may be unidentified. Fresh water from rivers or lakes, seawater from coastal areas, oceans; water in hot springs or thermal vents; and lake, marine, or estuarine sediments, can be used to source the algae. The algae may also be collected from local or remote bodies of water, including surface as well as subterranean water. Preferably, the algal species for use in the embodiments of the disclosure may be isolated from water, wastewater storage ponds, or soil that has been in contact with high volumes of industrial wastewater effluent for a prolonged period. This period of exposure will advantageously enrich the population of algae proliferating therein in those species and strains of algae able to utilize the wastewater as a nutrient source. It is not required that all the algae in an algal composition of the disclosure be taxonomically classified or characterized for the composition be used in the present disclosure. Algal compositions including algal cultures can be distinguished by the relative proportions of taxonomic groups that are present.

One or more species of algae may be present in the algal composition of the disclosure. In one embodiment of the disclosure, the algal composition is a monoculture, wherein only one species of algae is grown. However, in many open culturing systems, it may be difficult to avoid the presence of other algae species in the medium. Accordingly, a monoculture may comprise about 0.1% to 2% cells of algae species other than the intended species, i.e., up to about 98% to about 99.9% of the algal cells in a monoculture can be one species. In certain embodiments, the algal compositions may comprise an isolated species of algae, such as an axenic culture. In other embodiments, the algal composition can be a mixed culture that comprises more than one species of algae, i.e., the algal culture is not a monoculture. Such a culture can occur naturally with an assemblage of different species of algae or it can be prepared by mixing different algal cultures or axenic cultures. In certain embodiments, an algal composition comprising a combination of different batches of algal cultures is used in the disclosure. The algal composition can be prepared by mixing a plurality of different algal cultures. The different taxonomic groups of algae can be present in defined proportions. The combination and proportion of different algae in an algal composition can be designed or adjusted to yield a desired blend of algal lipids.

A mixed algal composition of the disclosure comprises one or several dominant species of macroalgae and/or microalgae. Microalgal species can be identified by microscopy and enumerated by counting, by microfluidics, or by flow cytometry, which are techniques well known in the art. A dominant species is one that ranks high in the number of algal cells, e.g., the top one to five species with the highest number of cells relative to other species. Microalgae occur in unicellular, filamentous, or colonial forms. The number of algal cells can be estimated by counting the number of colonies or filaments. Alternatively, the dominant species can be determined by ranking the number of cells, colonies and/or filaments. This scheme of counting may be preferred in mixed cultures where different forms are present and the number of cells in a colony or filament is difficult to discern. In a mixed algal composition, the one or several dominant algae species may constitute greater than about 10%, about 20%, about 30%, about 40%, about 50%, about 60%, about 70%, about 80%, about 90%, about 95%, about 97%, about 98% of the algae present in the culture. In certain mixed algal compositions, several dominant algae species may each independently constitute greater than about 10%, about 20%, about 30%, about 40%, about 50%, about 60%, about 70%, about 80% or about 90% of the algae present in the culture. Many other minor species of algae may also be present in such compositions but they may constitute in aggregate less than about 50%, about 40%, about 30%, about 20%, about 10%, or about 5% of the algae present. In various embodiments, one, two, three, four, or five dominant species of algae are present in an algal composition. Accordingly, a mixed algal culture or an algal composition can be described and distinguished from other cultures or compositions by the dominant species of algae present. An algal composition can be further described by the percentages of cells that are of dominant species relative to minor species, or the percentages of each of the dominant species. It is to be understood that mixed algal cultures or compositions having the same genus or species of algae may be different by virtue of the relative abundance of the various genus and/or species that are present. It is understood that for the purposes of the embodiments of the disclosure, the populations of algae, either monoculture or mixed populations are characterized as being able to proliferate on a medium comprising an industrial effluent wastewater, and optionally further comprising an amount of city sewage that allows growth of the algae to preferably increase over the growth rate in the absence of the added sewage. It is understood that the algal populations of the disclosure may be grown on untreated or treated wastewater. It is further understood that with a mixed population of algae, two or more of the species or strains of the mixed population may differ in their growth rates when cultured on the industrial wastewater-based media of the disclosure.

It should also be understood that in certain embodiments, such algae may be present as a contaminant, a non-dominant group or a minor species, especially in an open system. Such algae may be present in negligent numbers, or substantially diluted given the volume of the culture or composition. The presence of such algal genus or species in a culture, composition or a body of water is distinguishable from cultures, composition or bodies of water where such algal genus or species are dominant, or constitute the bulk of the algae. In various embodiments, one or more species of algae belonging to the following phyla can be used in the systems and methods of the disclosure: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta. In certain embodiments, algae in multicellular or filamentous forms, such as seaweeds and/or macroalgae, many of which belong to the phyla Phaeophyta or Rhodophyta, are less preferred.

In certain embodiments, the algal composition of the disclosure comprises cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

In certain embodiments, the algal composition of the disclosure may comprise, but is not limited to, algae from one or more of the following taxonomic classes: Euglenophyceae, Dinophyceae, and Ebriophyceae. Non-limiting examples include Euglena species and the freshwater or marine dinoflagellates.

In certain embodiments, the algal composition of the disclosure comprises, but is not limited to, green algae from one or more of the following taxonomic classes: Micromonadophyceae, Charophyceae, Ulvophyceae and Chlorophyceae. Non-limiting examples include species of Borodinella, Chlorella (e.g., C. ellipsoidea), Chlamydomonas, Dunaliella (e.g., D. salina, D. bardawil), Franceia, Haematococcus, Oocystis (e.g., O. parva, O. pustilla), Scenedesmus, Stichococcus, Ankistrodesmus (e.g., A. falcatus), Chlorococcum, Monoraphidium, Nannochloris and Botryococcus (e.g., B. braunii).

In certain embodiments, the algal composition of the disclosure comprises golden-brown algae from one or more of the following taxonomic classes: Chrysophyceae and Synurophyceae. Non-limiting examples include Boekelovia species (e.g. B. hooglandii) and Ochromonas species.

In certain embodiments, the algal composition in the disclosure may comprise freshwater, brackish, or marine diatoms from one or more of the following taxonomic classes: Bacillariophyceae, Coscinodiscophyceae, and Fragilariophyceae. Preferably, the diatoms are photoautotrophic, auxotrophic, or mixotrophic. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. muelleri, C. gracilis). Caloneis, Camphylodiscus, Cyclotella (e.g., C. cryptica, C. meneghiniana), Cricosphaera, Cymballa, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipata, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Surirella and Thalassiosira (e.g., T. weissflogii).

In certain embodiments, the algal composition of the disclosure comprises one or more algae from the following groups: Coelastrum, Chlorosarcina, Micractinium, Porphyridium, Nostoc, Closterium, Elakatothrix, Cyanosarcina, Trachelamonas, Kirchneriella, Carteria, Crytomonas, Chlamydamonas, Planktothrix, Anabaena, Hymenomonas, Isochrysis, Pavlova, Monodus, Monallanthus, Platymonas, Pyramimonas, Stephanodiscus, Chroococcus, Staurastrum, Netrium, and Tetraselmis.

Any named herein as being adapted for growth an industrial wastewater will be suitable for use in the aquaculture system and method of the disclosure including, but not limited to, a Chlamydomonas sp., Chlorella vulgaris, Chlorella sorokiniana, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp. Exemplary species include, by way of example and without limitation, microalgae such as Porphyridium cruentum, Spirulina platensis, Cyclotella nana, Dunaliella salina, Dunaliella bardawil, Phaeodactylum tricomutum, Muriellopsis spp., Chlorella fusca, Chlorella zofingiensis, Chlorella spp., Haematococcus pluvialis, Chlorococcum citriforme, Neospongiococcum gelatinosum, Isochrysis galbana, Chlorella stigmataphora, Chlorella vulgaris, Chlorella pyrenoidosa, Chlamydomonas mexicana, Scenedesmus obliquus, Scenedesmus braziliensis, Stichococcus bacillaris, Anabaena flos-aquae, Porphyridium aerugineum, Fragilaria sublinearis, Skeletonema costatum, Pavlova gyrens, Monochrysis lutheri, Coccolithus huxleyi, Nitzschia palea, Dunaliella tertiolecta, Prymnesium paruum, and the like.

The term “consortium” as used herein refers to a population of a plurality of algal species that are able to survive and proliferate using a culture medium, the culture medium comprising a treated or untreated wastewater effluent from an industrial or agricultural process combined with municipal commercial and residential sewage. The “consortium” may be assembled from isolates of algal species or isolated as a group of algal strains from a natural environment such as, but not limited to, a wasterwater holding reservoir. In such a case as a holding reservoir, it is contemplated that the isolated algal strains will be able to proliferate on the wastewater, although increases in their growth rates and biomass yields may be increased by subsequent genetic modification of by additions or modifications to the culture medium. The term “primary consortium” as used herein refers to a population of algal strains initially isolated from a medium enriched in an industrial wastewater and inoculated with isolates from a storage pond or a location subject to prolonged exposure to an industrial wastewater. In one example, the wastewater can be from the carpet manufacturing industry. Most advantageously for use in the methods of the disclosure the consortium can comprise three strains of algae: Chlamydomonas globosa, Chlorella minutissima, and Scenedesmus bijuga, and optionally a fourth strain, Chlorella sorokiniana.

The term “biomass” as used herein refers to the mass and/or accumulating mass of photosynthetic organisms resulting from the cultivation of such organisms using a variety of techniques.

The terms “photobioreactor,” “photobioreactor apparatus”, or “reactor” as used herein refer to an apparatus containing, or configured to contain, a liquid medium comprising at least one species of photosynthetic organism and having either a source of light capable of driving photosynthesis associated therewith, or having at least one surface at least a portion of which is partially transparent to light of a wavelength capable of driving photosynthesis (i.e. light of a wavelength between about 400-700 nm). Certain photobioreactors for use herein comprise an enclosed bioreactor system such as, but not limited to, a polybag, as contrasted with an open bioreactor, such as a pond or other open body of water, open tanks, open channels such as a raceway, and the like.

The term “raceway” as used herein refers to elongated (long and narrow) tanks or liquid paths that provide a flow-through system for a culture medium, thereby enabling a higher yield of biomass than would be achieved by a static pond system.

The term “biofuel” as used herein refers to an organic fuel derived from biomass. The term “biomass” encompasses solid biomass, liquid fuels and various biogases. Bioethanol is an alcohol (ethanol) made by fermenting the sugar components of plant materials and has been made mostly from sugar and starch crops. With advanced technology being developed, cellulosic biomass, such as trees and grasses are also used as feedstocks for ethanol production. Ethanol can be used as a fuel for vehicles in its pure form, but it is usually used as a gasoline additive. The predominant biogas produced from a biomass is typically methane but may also include minor percentages of other alkyl-chain gases and volatile compounds.

The term “biodiesel” as used herein refers to a vegetable oil- or animal fat-based diesel fuel comprising long-chain alkyl (methyl, propyl or ethyl) esters. Biodiesel is typically made by chemically reacting lipids, such as derived from algae cultured by the methods of the present disclosure, with an alcohol. Biodiesel can be produced from oils or fats using transesterification. Biodiesel is meant to be used in standard diesel engines and is distinct from the vegetable and waste oils. Biodiesel can be used alone, or blended with petrodiesel. The term “biodiesel” can be standardized as mono-alkyl ester in the United States.

Generally, a process for production of biofuels from algae can include cultivating oil-producing algae by promoting both autotrophic and heterotrophic growth. Heterotrophic growth can include introducing an algal feed to the oil-producing algae to increase the formation of algal oil. The algal oil can be extracted from the oil-producing algae using biological agents and/or other methods such as mechanical pressing. The resulting algal oil can be subjected to a transesterification process to form biodiesel.

The terms “transesterify,” “transesterifying,” and “transesterification” refer to a process of exchanging an alkoxy group of an ester by another alcohol and more specifically, of converting algal oil, e.g. triglycerides, to biodiesel, e.g. fatty acid alkyl esters, and glycerol. Transesterification can be accomplished by using traditional chemical processes such as acid or base catalyzed reactions, or by using enzyme-catalyzed reactions.

### Discussion

The embodiments of the present disclosure incorporate the robustness of flora isolated from environments exposed to the type of effluent to be encountered when the algae are cultured on industrial wastewater and are resistant to local climatic changes and the fluctuating extreme environments of the wastewaters that may be used for their cultivation. Mixotrophic forms that provide greater biomass and lipid yields than do obligate photoautotrophic algae are preferred in the methods of the disclosure. Even when such algae have low lipid content, their high productivity can compensate. Mixotrophic algae such as, but not limited to, Chlorella minutissima, Chlorella sorokiniana, Chlamydomonas globosa and Scenedesmus bijuga, either individually or as a consortium of these strains can be used for culturing in municipal wastewater, poultry litter extract in water, and untreated and treated industrial wastewater, and the like. Accordingly, the embodiments of the present disclosure encompass, among other aspects, mixed algal populations able to survive and proliferate on culture media that have a high proportion of an industry wastewater. The embodiments of the disclosure further encompass methods of mixotrphically cultivating mixed populations of freshwater and marine algae comprising a plurality of genera and species to provide a biomass from which may be extracted lipids, or be converted into biodiesel by such procedures as pyrolysis. Lipid material extracted from the algae may be converted to biodiesel or other organic products.

Industrial wastewaters show wide variation in quality. A stream of an agricultural industry (poultry industry) untreated wastewater and combined with 10%-15% sewage i.e. a municipal wasterwater that has not been passed through a water treatment plant, has been found to be a good growth medium for cultivation of microalgae. Algal biomass and biodiesel production using a wastewater containing between about 85% to about 90% carpet industry effluents treated with 10%-15% municipal sewage was shown. Growth studies indicated both fresh water and marine algae showed good growth in wastewaters.

A consortium of native algal isolates showed more than 96% removal of nutrients from treated wastewater and provided potential scaled-up biomass production of approximately 9.2-17.8 tons per hectare per annum. The lipid content of this consortium when cultivated in treated wastewater was approximately 7% wt/wt. About 65% of the algal oil obtained from the consortium could be converted into biodiesel.

Wastewater bioremediation by microalgae provides several advantages as it is an eco-friendly process with no secondary pollution, if the biomass produced is reused; and it allows efficient nutrient recycling (Oswald W. J. (1963) Dev. Ind. Microbiol. 4: 112-119; Olguin E. J. (2003) Biotechnol. Adv. 22: 81-91). Algae are microorganisms capable of performing photosynthesis more efficiently than plants using sunlight and carbon dioxide The potential biomass productivity of algae under optimum scenario ranges from about 100 to about 150 tons per hectare per annum (Rodolfi et al., (2008) Biotechnol. Bioeng. 102: 100-112; Weyer et al., (2009) Bioenerg. Res. DOI 10.1007/s12155-009-9046-x), a factor 10-15 times higher than the productivity of conventional agricultural crops. Algae do not need soil and can grow in poor quality wastewaters.

Algae have the potential to produce about 40,700-53,200 liters per hectare per annum of oil (Weyer et al., (2009) Bioenerg. Res. DOI 10.1007/s12155-009-9046-x), which is 6 to 8 times better than the yield of oil palm considered currently the best source for the purpose. Oil from algae can be used for biodiesel while residual biomass can be fermented into ethanol and biomethane.

Biofuels derived from plants like algae are considered “carbon neutral”. Two of the most limiting factors to a sustainable and economic production of algae for biofuel purposes are water and fertilizers. Maximum cultivation of algae would require 2 million liters of water per hectare if grown in open ponds, but to compensate for evaporative losses a further 11 million liters would be required. Hence, water management is a critical bottleneck in practical algae cultivation.

Cultivation of algae can also require supplementation of nutrients, particularly nitrogen and phosphorus. Increasing fertilizer costs make economically feasible production of algae a still difficult target. The methods of use of wastewater generated by an industry, combined with a typical city sewage, as encompassed by the disclosure, provides a cheap source of an algal culture medium while simultaneously being treated to reduce or remove the industry by-products that are undesired in the environment.

The present disclosure, therefore, provides isolated cultures of algae that show the capacity to survive and proliferate on the wastewater, particularly that derived from agricultural industry, and methods of use thereof. In particular, embodiments of the disclosure provide mixed populations of algal mixotrophs that provide growth rates and growth yields that are suitable for the economic production of algal biomass and biodiesel therefrom.

Although the isolated algae and combinations thereof according to the disclosure are able to grow on agricultural industry wastewater under a variety of conditions, the embodiments of the disclosure further provide a system for the algal cultivation that overcomes some, at least, of the inherent disadvantages of industry wasterwater such as, but not limited to, a carpet industry, as a culture medium, and especially the prescence of dyes and other colorants that reduce the amount of illumination reaching the algae.

The production of energy in the form of oil (lipids) by algae is more useful than the production of starch. If equal volumes of oil and starch are produced, the oil will contain significantly more energy. For example, the energy content in a typical algal lipid is 9 kcal/gram compared to 4.2 kcal/gram for typical algal starch. In the production of sugars from starch, not all the starch is saccharified into sugars which can be easily fermented, so a portion may be lost as unused sugars. Also, the production of biodiesel from the algal oil is essentially energy-neutral, so nearly all of the energy content of the algal oil is retained in the biodiesel. In contrast, the production of alcohol from biomass or starch is less efficient, especially during the fermentation stage which converts the sugars derived from the biomass or starch into alcohol. Fermentation is exothermic, with heat being generated that must be removed and often wasted. One half of the carbon in the sugar is released during fermentation as carbon dioxide and is therefore not available for fuel energy. Distillation of the ethanol is an energy-dependent process. Thus, biodiesel production is more efficient overall than bioethanol production, and therefore the goal of highest efficiency and lowest cost is served by maximizing biodiesel production.

Nevertheless, starch-producing or biomass-producing algae are significant aspects of the present disclosure and biomass production can be economically significant. For example, starch products or sugars converted from algal biomass can be used to produce feed for the oil-producing algae and/or production of ethanol or ethyl acetate for use in transesterification of algal oil. Carbon dioxide released during fermentation can be fed back into the algal growth stage, substantially eliminating at least this form of energy loss in the fermentation process.

Any one or more methods for dewatering an algal biomass can be used including but not limited to, sedimentation, filtration, centrifugation, flocculation, froth floatation, and/or semi-permeable membranes, which can increase the concentration of algae by a factor of about 2, 5, 10, 20, 50, 75, or 100. The dewatering step can be performed serially by one or more different techniques to obtain a concentrated algal composition before extraction of lipids therefrom or before fermentation, pyrolysis and the like for the generation of a biofuel. See, for example, Chapter 10 in Handbook of Microalgal Culture, edited by Amos Richmond, 2004, Blackwell Science, for description of downstream processing techniques. Centrifugation separates algae from the culture media and can be used to concentrate or dewater the algae. Various types of centrifuges known in the art, including but not limited to, tubular bowl, batch disc, nozzle disc, valve disc, open bowl, imperforate basket, and scroll discharge decanter types, can be used. Filtration by rotary vacuum drum or chamber filter can be used for concentrating fairly large microalgae. Flocculation is the collection of algal cells into an aggregate mass by addition of polymers, and is typically induced by a pH change or the use of cationic polymers. Foam fractionation relies on bubbles in the culture media which carries the algae to the surface where foam is formed due to the ionic properties of water, air and matter dissolved or suspended in the culture media. An algal composition of the disclosure can be a concentrated algal culture or composition that comprises about 110%, 125%, 150%, 175%, 200% (or 2 times), 250%, 500% (or 5 times), 750%, 1000% (10 times) or 2000% (20 times) the amount of algae in the original culture or in a preceding algal composition. An algal composition can also be described by its moisture level or level of solids, especially when it is in a paste form, such as but not limited to a paste comprising about 1%, 2%, 5%, 10%, 15%, 20%, 25%, 30%, 35%, 40%, 45%, or 50% solids by weight.

Mechanical crushing, for example, an expeller or press, a hexane or butane solvent recovery step, supercritical fluid extraction, or the like can also be useful in extracting the oil from oil vesicles of the oil-producing algae grown using the methods of the disclosure. Alternatively, mechanical approaches can be used in combination with biological agents in order to improve reaction rates and/or separation of materials.

Once the oil has been released from the algae it can be recovered or separated from a slurry of algae debris material, e.g. cellular residue, oil, enzyme, by-products, etc. This can be done by sedimentation or centrifugation, with centrifugation generally being faster. Starch production can follow similar separation processes. Recovered algal oil can be collected and directed to a conversion process. The algal biomass left after the oil is separated may be fed into the depolymerization stage described below to recover any residual energy by conversion to sugars, and the remaining husks can be either burned for process heat or sold as an animal food supplement or fish food.

Algal oil can be converted to biodiesel through a process of direct hydrogenation or transesterification of the algal oil. Algal oil is in a similar form as most vegetable oils, which are in the form of triglycerides. This form of oil can be burned directly. However, the properties of the oil in this form are not ideal for use in a diesel engine, and without modification, the engine will soon run poorly or fail. In accordance with the present disclosure, the triglyceride is converted into biodiesel, which is similar to but superior to petroleum diesel fuel in many respects.

One process for converting the triglyceride to biodiesel is transesterification, and includes reacting the triglyceride with alcohol or other acyl acceptor to produce free fatty acid esters and glycerol. The free fatty acids are in the form of fatty acid alkyl esters. Transesterification can be done in several ways, including biologically and/or chemically. The biological process uses an enzyme known as a lipase to catalyze the transesterification, while the chemical process may use, but is not limited to, a synthetic catalyst that may be either an acid or a base. With the chemical process, additional steps are needed to separate the catalyst and clean the fatty acids. In addition, if ethanol is used as the acyl acceptor, it must be essentially dry to prevent production of soap via saponification in the process, and the glycerol must be purified. Either or both of the biological and chemically-catalyzed approaches can be useful in connection with the processes of the present disclosure.

Algal triglyceride can also be converted to biodiesel by direct hydrogenation. In this process, the products are alkane chains, propane, and water. The glycerol backbone is hydrogenated to propane, so there is substantially no glycerol produced as a byproduct. Furthermore, no alcohol or transesterification catalysts are needed. All of the biomass can be used as feed for the oil-producing algae with none needed for fermentation to produce alcohol for transesterification. The resulting alkanes are pure hydrocarbons, with no oxygen, so the biodiesel produced in this way has a slightly higher energy content than the alkyl esters, degrades more slowly, does not attract water, and has other desirable chemical properties.

Accordingly, one aspect of the present disclosure encompasses methods of generating an algal biomass, comprising: (a) forming an algal culture by combining: (i) a population of algal cells characterized as proliferating in a culture medium comprising an industry wastewater; (ii) a culture medium comprising an industry wastewater, optionally a municipal sewage effluent, and optionally a nutritional supplement, where said nutritional supplement increases the yield of algal culture compared to when the culture medium does not comprise the nutritional supplement, said nutritional supplement comprising an organic carbon source suitable for supporting the proliferation of a mixotrophic algal species, a mineral, a buffer, or a combination thereof; and (b) maintaining the algal culture under conditions suitable for the proliferation of the population of algal cells, thereby forming an algal biomass.

In embodiments of this aspect of the disclosure, the industry wastewater can be the effluent from an agricultural industry.

In other embodiments of the methods of this aspect of the disclosure, the effluent can be from a poultry industry, a non-poultry meat industry, or a plant-based industry.

In yet other embodiments, the industry wastewater can be obtained from a non-agricultural industry.

In the embodiments of this aspect of the disclosure, the nutritional supplement can comprise at least one organic carbon source selected from the group consisting of: glucose, sucrose, arabinose, fructose, glycerol, methanol, acetate, a plant-based hydrolyzate, and any combination thereof.

In the embodiments of this aspect of the disclosure, the population of algal cells can comprise a freshwater (non-marine) algal strain, a plurality of freshwater (non-marine) algal strains, a plurality of cyanobacter strains, a plurality of diatomaceous algal strains, or any combination thereof, where at least one species of the population of algal cells is a mixotrophic alga.

In the embodiments of this aspect of the disclosure, at least one algal strain of the population of algal cells may be isolated from a source in contact with the wastewater effluent of an industry.

In some embodiments of this aspect of the disclosure, the population of algal cells can comprise a strain of an algal genus selected from the group consisting of: Scenedesmus, Chlorella, Chlamydomonas, Scenedesmus and Chorella, Scenedesmus and Chlamydomonas, Chorella and Chlamydomonas, and Scenedesmus, Chorella, and Chlamydomonas.

In embodiments of this aspect, at least one algal strain of the population of algal cells can be selected from the group consisting of: a Chlamydomonas sp., Chlorella vulgaris, Chlorella sorokiniana, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

In certain embodiments of this aspect of the disclosure, the population of algal cells can be a consortium of algal cells comprising Chlamydomonas globosa, Chlorella minutissima, and Scenedesmus bijuga, and optionally Chlorella sorokiniana.

In embodiments of this aspect of the disclosure, the methods can further comprise isolating the algal biomass from the medium.

Yet another aspect of the disclosure encompasses methods of producing a biofuel from industrial wastewater comprising: (a) forming an algal culture by combining: (i) a population of algal cells characterized as proliferating in a culture medium comprising an industry wastewater; (ii) a culture medium comprising an industry wastewater, optionally a municipal sewage effluent, and optionally a nutritional supplement, where the nutritional supplement increases the yield of algal culture compared to when the culture medium does not comprise the nutritional supplement, the nutritional supplement comprising an organic carbon source suitable for supporting the proliferation of a mixotrophic algal species, a mineral, a buffer, or a combination thereof, and where when the industry wastewater is an agricultural industry effluent, the agricultural industry is a poultry industry, a non-poultry meat industry, or a crop-based industry; (b) maintaining the algal culture under conditions suitable for the proliferation of the population of algal cells, thereby forming an algal biomass; (c) isolating the algal biomass from the medium; and (d) obtaining from the isolated algal biomass a biofuel or a source of a biofuel, where the step of obtaining from the isolated biomass a biofuel comprises the steps of isolating a lipid material from the biomass or converting the biomass to a biofuel, and where the isolated lipid material is converted to a biofuel.

In embodiments of this aspect of the disclosure, the nutritional supplement can comprise at least one organic carbon source selected from the group consisting of: glucose, sucrose, arabinose, fructose, glycerol, methanol, acetate, a plant-based hydrolyzate, and any combination thereof.

In the embodiments, the population of algal cells may comprise a freshwater (non-marine) algal strain, a plurality of freshwater (non-marine) algal strains, a plurality of cyanobacter strains, a plurality of diatomaceous algal strains, or any combination thereof, and at least one algal strain of the population of algal cells is isolated from a source in contact with the wastewater effluent of an industry.

In certain embodiments, the population of algal cells may comprise an algal genus selected from the group consisting of: Scenedesmus, Chlorella, Chlamydomonas, Scenedesmus and Chlorella, Scenedesmus and Chlamydomonas, Chorella and Chlamydomonas, and Scenedesmus, Chorella, and Chlamydomonas.

In some embodiments of the disclosure, at least one algal strain of the population of algal cells can be selected from the group consisting of: a Chlamydomonas sp., Chlorella vulgaris, Chlorella sorokiniana, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

In one embodiment, the population of algal cells can be a consortium of algal cells comprising Chlamydomonas globosa, Chlorella minutissima, and Scenedesmus bijuga, and optionally Chlorella sorokiniana.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

Three green algae, Chlamydomonas globosa, Chlorella minutissima, and Scenedesmus bijuga were isolated and maintained in BG 11 medium. Six marine algae, Dunaliella parva, D. tertiolecta, Tetraselmis chuii, T. suecica, Phaeodactylum tricomutum—a diatom, and Pleurochrysis carterae—a Coccolithophorid, were maintained in seawater BG11 medium at 25° C.±2° C. temperature and illuminated with between 80-100 μmoles s⁻¹ light intensity in a 12 h-12 h light:dark cycle.

Poultry litter was obtained from a broiler farm and kept in ziplock bags in a cooler until used. Treated and untreated community wastewater was obtained from a North Georgia city water treatment plant and plant hydrolysates were from Michigan State University. Untreated community wastewater from the utility company contained approximately 85-90% wastewater from local carpet mills. All wastewaters were kept in coolers until used.

### Example 2

Isolation of mixotrophic algae: An enrichment of algal isolated from carpet wastewater was carried out in BG 11 medium and was then maintained by frequent subculturing under 80-100 μmoles m⁻² s⁻¹ light intensity in 12 h-12 h light/dark cycles and 25° C.±1° C. This enrichment was used to inoculate 200 gallons capacity (750 L) raceway ponds to run with wastewater throughout the year.

Continuous subculturing led to domination of the community by a limited number of species of Chlamydomonas, Scenedesmus and Chlorella. The mixture growing in the raceway ponds were inoculated in carpet industry treated wastewater with and without glucose and sucrose supplementation. Absorbance at 750 nm was used for growth measurements. The flasks showing higher absorbance in the presence of glucose were used to isolate algal cultures. Their purity was confirmed microscopically and they were identified using standard taxonomic keys. Subsequently these purified unialgal forms were maintained by frequent subculturing in BG 11 medium.

To determine whether organisms could grow heterotrophically under dark conditions and perform mixotrophic metabolism, they were cultured in 100 ml BG 11 medium with or without 1% glucose in 250 ml Erlenmeyer flasks. Three such flasks with each type of medium for each algal strain were wrapped completely with aluminium foil to create dark conditions and a similar set was kept in a 12 h-12 h light/dark cycle for 7 days. Light intensity was 80-100 μmoles m⁻² s⁻¹. After 7 days of incubation, growth was observed in terms of both chlorophyll a and biomass.

### Example 3

Establishing heterotrophy and mixotrophy: The process of extraction of nutrients from poultry litter generated a dark color in water that reduced by 92% the light at a depth of 4.3 cm water. Colored wastewaters would be expected to have adverse effects on photosynthesis. Thus it was considered important that the algae that could be grown in poultry litter extract be mixotrophic and able to metabolize both inorganic and organic carbon substrates in the presence of light, and that the processes of autotrophy and heterotrophy not inhibit each other.

Algae, therefore, were grown in BG11 medium supplemented with or without 1% glucose. After inoculations, three flasks of each with glucose were wrapped with aluminium foil to stop light penetration. All were incubated for 10 days. Several strains were tested and three strains were selected based on their performance.

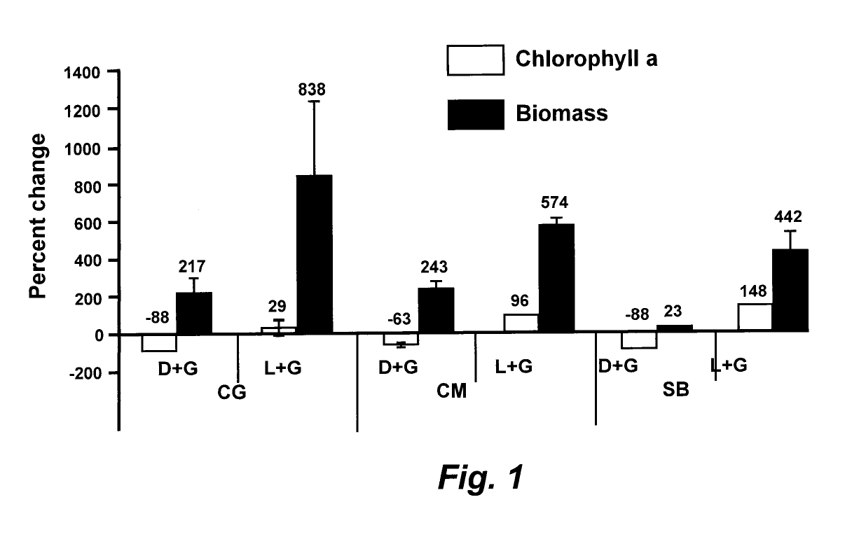

All three of the test algae, Scendesmus bijuga, Chlamydomonas globosa, and Chlorella minutissima grew in the absence of light and glucose, with 89%, 74%, and 197% increases in chlorophyll a, respectively. However, this heterotrophic growth was about 62% to about 88% less than that under phototrophic conditions, i.e. in presence of light but with no glucose. When grown over glucose in presence of light, the best response was that of Scenedesmus bijuga (a 148% increase over phototrophic growth) followed by Chlorella minutissima (a 96% increase), while Chlamydomonas resulted in only a 29% increase in chlorophyll a, as shown in FIG. 1.

Chlamydomonas globosa, with light+glucose (mixotrophy) gave about 10 times more biomass increase than without glucose; however, glucose in the dark resulted in about 3 times less growth than did glucose plus light conditions (FIG. 1).

The Scenedesmus bijuga biomass yield was 5 times greater in the presence of light and glucose and almost equal to that between light without glucose (photoautotrophic) and dark+glucose (chemoheterotrophic). With Chlorella minutissima, biomass in dark+glucose and light+glucose conditions was 3 and 7 times more, respectively, than the biomass content in light without glucose (phototrophy).

The chlorophyll a content was higher in light+glucose than in dark+glucose in all the three test algae, as shown in FIG. 1. The biomass as well as the amount of chlorophyll a in light+glucose was more than the sum ([dark+glucose]+[light-glucose]), indicating that the three algae were capable of growing mixotrophically.

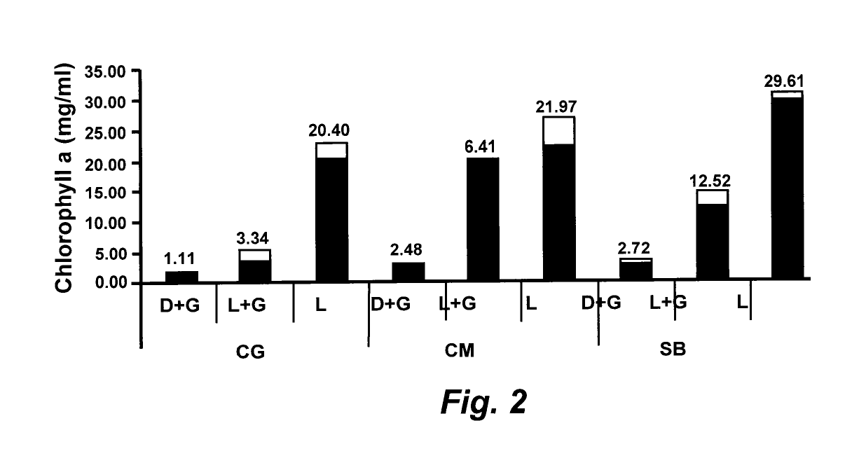

A feature of mixotrophy/heterotrophy was the reduction in overall chlorophyll content. In the three test algae, mixotrophy reduced by 15.6% to 17% the chlorophyll in cells over phototrophic growth, while heterotrophy further reduced by about 2-10% of total chlorophyll over mixotrophic growth, as shown in FIG. 2.

### Example 4

Organic carbon substrate utilization profile: Glucose, sucrose, acetate (sodium acetate), methanol, and glycerol were used at 1% w/v concentrations each to determine if they could be utilized by the isolated algae in presence or absence of light using the culture methods as described in Example 3, above.

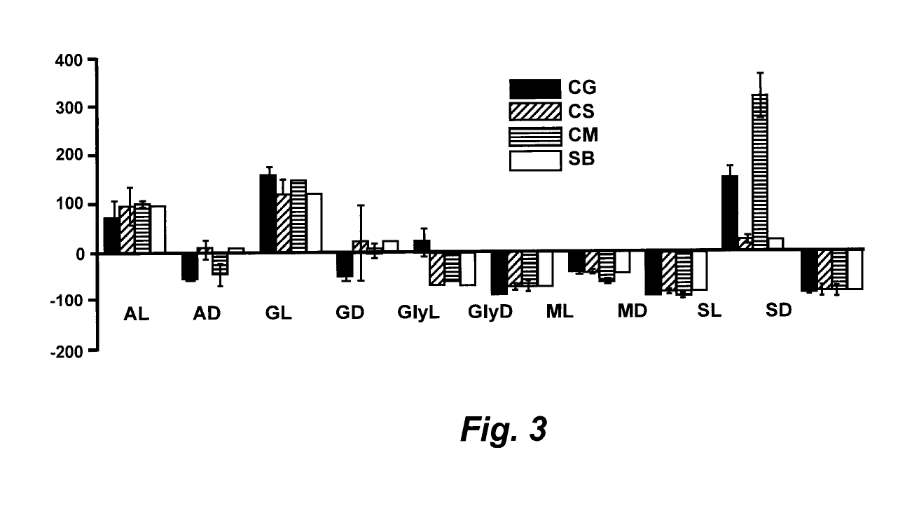

Methanol was used under illuminated conditions, but growth was reduced by 40% for Chlamydomonas globosa, Chlorella sorokiniana, and Scenedesmus bijuga, and 61% for Chlorella minutissima, when compared to growth on BG11. Under dark conditions, growth was suppressed by about 82% to about 90%, as shown in FIG. 3. With glycerol, growth suppression in the dark was about 71% to about 85%, the maximum being with Chlamydomonas globosa. However, the same alga recorded a 21% increased growth with BG 11 over glycerol with light.

Heterotrophic growth with sucrose was about 78% to about 83% less than photoautotrophic growth on BG 11 medium, while C. sorokiniana and S. bijug showed 22% increases under mixotrophic conditions, C. globosa showed about 147% growth yield increase over BG 11 medium, while a maximum of about 311% increase was observed with C. minutissima.

With acetate, C. globosa and C. minutissima showed about 50% and 43% growth reductions as compared to when under phototrophic conditions, but C. sorokiniana and S. bijuga had 8% greater chlorophyll a, while under mixotrophic conditions, all showed about 73% to 99% increases. Glucose supported about 47% less growth of C. globosa under heterotrophic conditions, but others had only about 3% to 21% more chlorophyll a than under phototrophic conditions. Mixotrophy yielded about 119% to 156% increases in growth in terms of chlorophyll a in all algae tested, as shown in FIG. 3.

### Example 4

Poultry litter extract preparation: Experiments showed that 1.25% (w/v) poultry litter tied in cotton bags hung in deionized water being stirred with magnets for 1 hour at room temperature could yield sufficient nutrients (10-50 mg/L of nitrate and ammonia nitrogen, and 7-20 mg/L of phosphate) to support growth of the three algae to yield algae increases equal to or greater than that obtainable using the standard growth medium-BG 11. Therefore, the same amount of poultry litter was used to extract nutrients by replacing deionized water with different wastewaters being tested.

### Example 5

Growth in various wastewater sources: A water extract of poultry litter (1.25%) and carpet industry treated and untreated water (comprising of 85-90% effluents from carpet industries combined with 10-15% sewage) were used to judge the performance of mixotrophic isolates in comparison with growth on BG 11 medium.

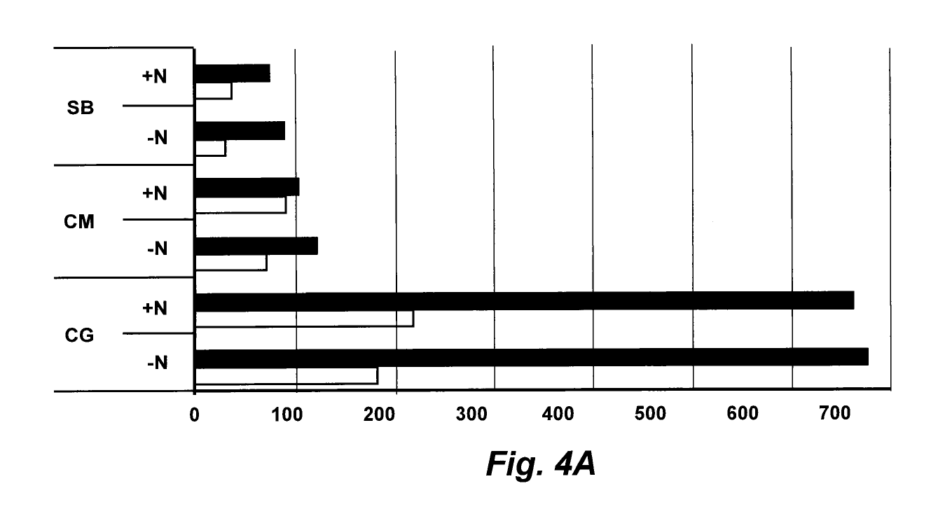

All the four treatments, i.e. carpet industry treated and untreated wastewater, poultry litter extract, and BG 11 medium, were dispensed as 100 mls of each in triplicates of 250 ml capacity Erlenmeyer flasks for the alga Chlamydomonas globosa BCCP 101, Chlorella minutissima BCCP 102, and Scenedesmus bijuga BCCP 103. Ten ml of growing culture (6 day old) of each algal strain was inoculated to each flask. After 5 days of incubation under standard conditions, biomass, chlorophyll a, and chlorophyll b were determined. The results are shown in FIGS. 4A-4C.

The three mixotrophic isolates, C. globosa, C. minutissima, and S. bijuga were also grown in treated and untreated carpet wastewater and in water extract of poultry litter (1.25% w/v), the results were compared with the growth in BG 11 medium. Since the wastewaters contained less nitrogen (5.1 ppm, 26.58 ppm, and 36.27 ppm, respectively, in carpet industry treated, untreated wastewater, and poultry litter extract), a nitrogen-supplemented treatment was also included. All treatments in triplicates were incubated in a growth room under conditions cited above for a period of 5 days.

Poultry litter extract stimulated a better growth response compared with BG 11 medium with all the three algae. But the stimulation was maximum with C. globosa where chlorophyll a showed more than 660% increase and biomass showed over 180% increase compared with that using BG11. It also recorded more than 260% increase in chlorophyll a and above 160% increase in biomass in carpet industry treated water, but could not survive in untreated carpet industry water.

Despite the dark colour of water, particularly in case of poultry litter extract, but also with untreated wastewater from the carpet industry, Scenedesmus bijuga and Chlorella minutissima (except in carpet industry treated water with no additional nitrogen) could grow better than on BG 11 medium alone, even without supplementation of additional nitrogen to the wastewater (FIG. 4A). Chlamydomonas globosa, however, could not survive in carpet industry untreated wastewater, but showed the growth improvement in two other wastewaters. Except for Chlamydomonas globosa in poultry litter extract, the other two algal strains showed growth improvement in wastewaters that was at similar to the improvement shown by them in nitrogen-supplemented wastewater. Carpet industry treated and untreated wastewaters were the best for Scenedesmus bijuga, and poultry litter extract followed by carpet industry treated water were the better for Chlamydomonas globosa.

C. minutissima, grown on carpet industry untreated water recorded greater than a 28% increase in chlorophyll a and greater than a 50% increase in biomass compared to when grown on BG 11 medium. On treated water from the carpet industry, however, the yield was equal to, or 32% better than that with BG 11 medium in terms of biomass generation, whereas chlorophyll a showed up to a 46% increase (see FIG. 4B). In terms of chlorophyll a, S. bijuga gave a similar yield with BG 11 medium or carpet industries untreated water, but biomass increase was significantly high (greater than 40%), as shown in FIG. 4C.

The addition of extra nitrogen (36.27 ppm nitrogen) in poultry litter extract did not yield any significant change in growth stimulation of all three algae. In carpet industry treated water (5.1 ppm nitrogen), S. bijuga showed no significant change in chlorophyll a improvement but the biomass production was better in carpet industry-treated water without added nitrogen. C. minutissima showed better growth enhancement on addition of nitrogen to carpet industry-treated water. A significant enhancement in chlorophyll a as a result of nitrogen supplementation in C. globosa did not be translated into proportionate increase in biomass. As a result the later was similar under both treatments.

### Example 6

Growth of algal consortia in wastewaters compared to BG 11 medium: Growing phase algae (6 days old) were inoculated as mixtures of 1:1 ratios of Chlamydomonas globosa:Chlorella minutissima; Chlamydomonas globosa: Scenedesmus bijuga; Chlorella minutissima:Scenedesmus bijuga; or a mixture (in a 1:1:1 ratio) of 6 day-old Chlamydomonas globosa:Chlorella minutissima: Scenedesmus bijuga. Individual algae types were also inoculated.

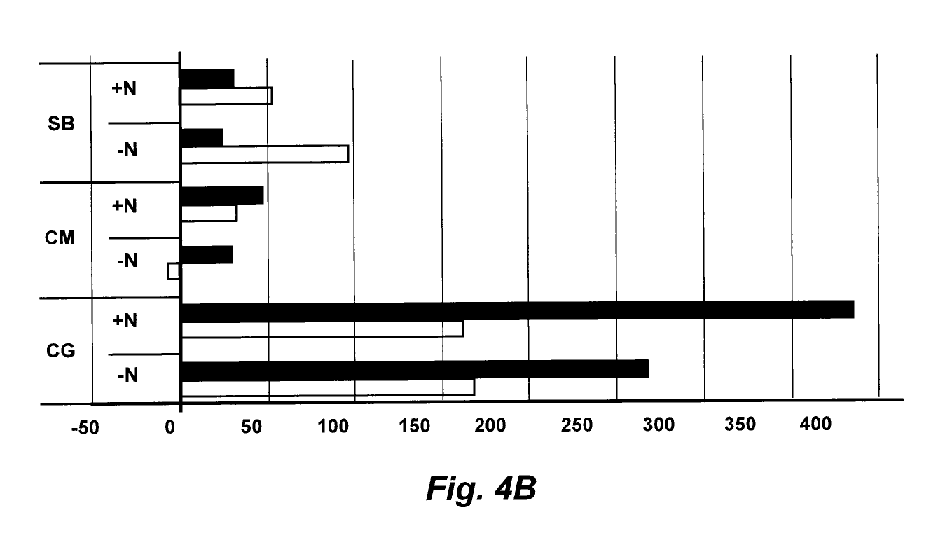

After 5 days of incubation, biomass, chlorophyll a and chlorophyll b were determined. A combination of all the three test alga showed less than 20% biomass generation than in BG 11 medium both in the presence and absence of additional nitrogen, as shown in FIG. 5. The two combinations of C. globosa-C. minutissima and S. bijuga-C. minutissima showed that the addition of nitrogen did not lead to significant increases in growth over those wastewaters that had not been so supplemented. The Chlamydomonas-Chlorella combination was the best for poultry litter extract, while the Scenedesmus-Chlorella combination was the best for carpet industry-untreated wastewater (FIG. 5).

### Example 7

### Growth Performance in Growth Medium Supplemented with Lignocellulosic Plant Hydrolysates

The growth responses of the mixotrophic algal strains Chlorella sorokiniana, Chlorella minutissima, Chlamydomonas globosa and Scenedesmus bijuga were assessed in a growth medium containing concentrations of 0%, 5%, 10%, and 15% of cornstalk lignocellulosic hydrolysate (Table 1) containing 60 g/L of glucose in BG11 medium or deionized water derived from AFEX process.

TABLE 1
Organic carbon compounds present in the growth medium on day 0 supplemented with
different concentrations of plant hydrolysates

| Hydrolysate | Glucose | Xylose | Arabinose | Succinate | Lactate | Formate | Acetate | Ethanol |
|---|---|---|---|---|---|---|---|---|
| conc. | (g/L) | (g/L) | (g/L) | (g/L) | (g/L) | (g/L) | (g/L) | (g/L) |
| BG5% | 0.67 | 0.41 | 0.10 | BDL | 0.07 | 0.02 | 0.04 | 0.21 |
| DI5% | 0.61 | 0.48 | 0.11 | BDL | BDL | BDL | 0.04 | 0.06 |
| BG10% | 4.06 | 1.76 | 0.45 | BDL | 0.02 | BDL | 0.11 | 0.11 |
| DI10% | 4.22 | 1.96 | 0.48 | BDL | 0.07 | BDL | 0.13 | 0.11 |
| BG15% | 6.21 | 2.93 | 0.74 | BDL | 0.06 | BDL | 0.16 | 0.26 |
| DI15% | 7.03 | 3.24 | 0.78 | BDL | 0.10 | BDL | 0.19 | 0.15 |

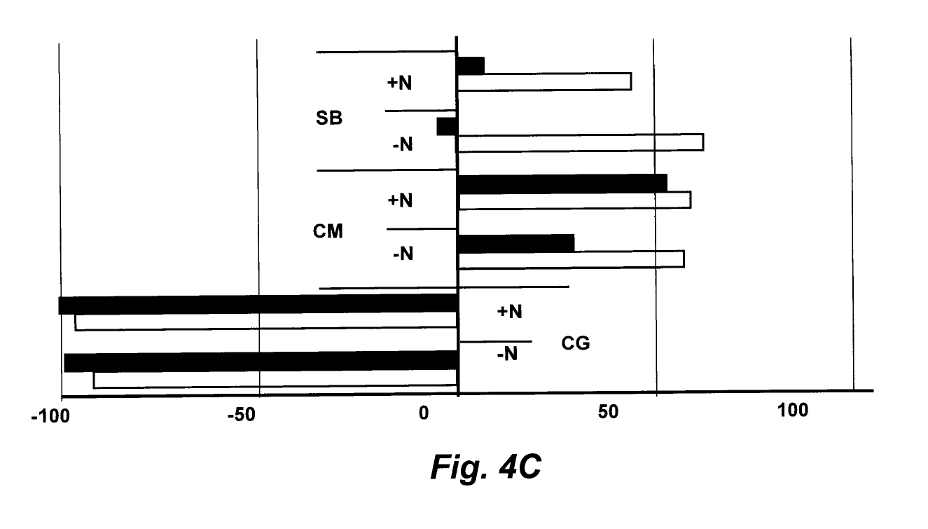

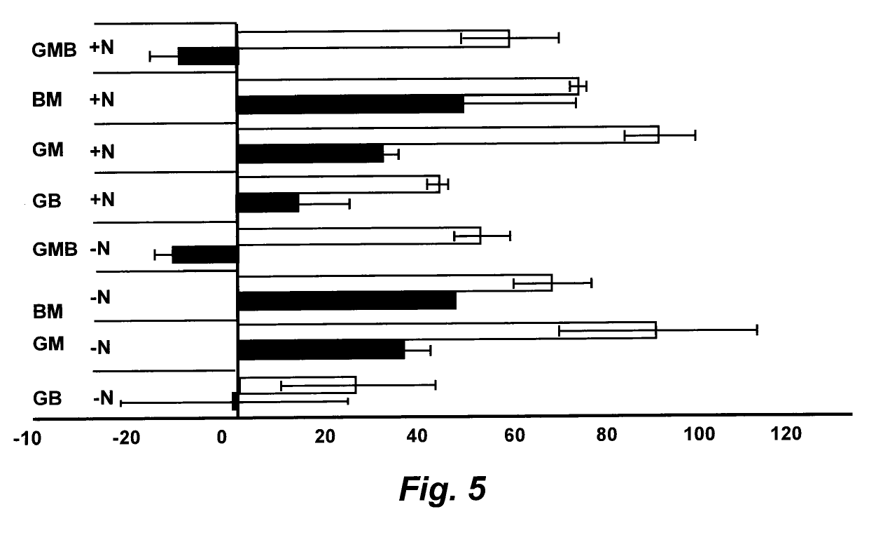

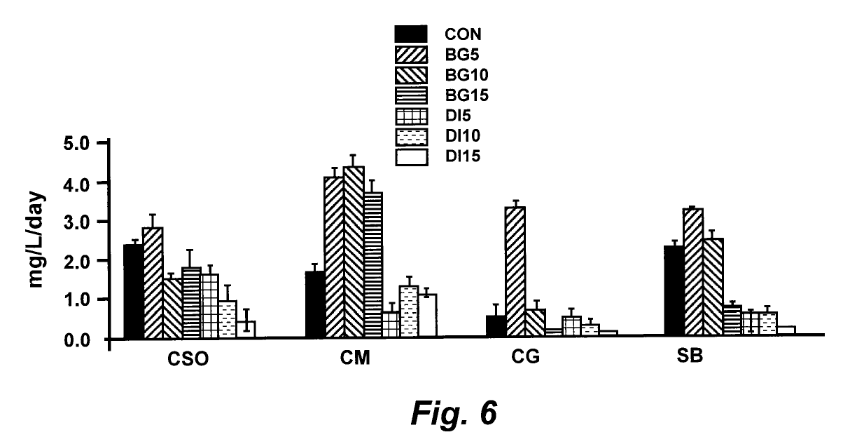

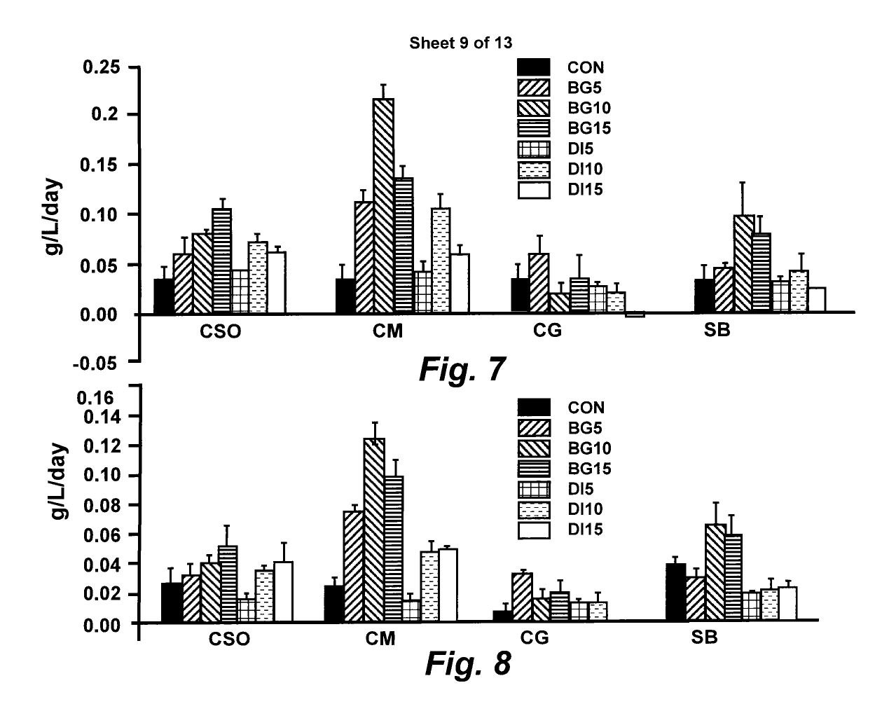

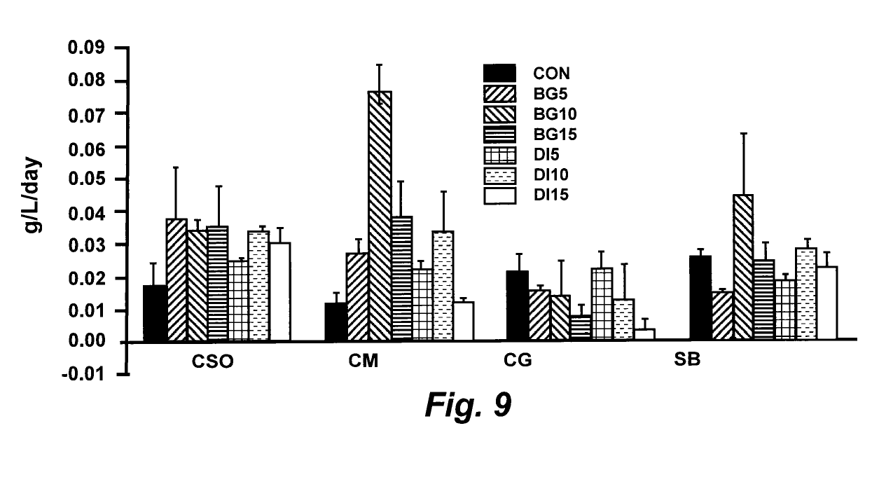

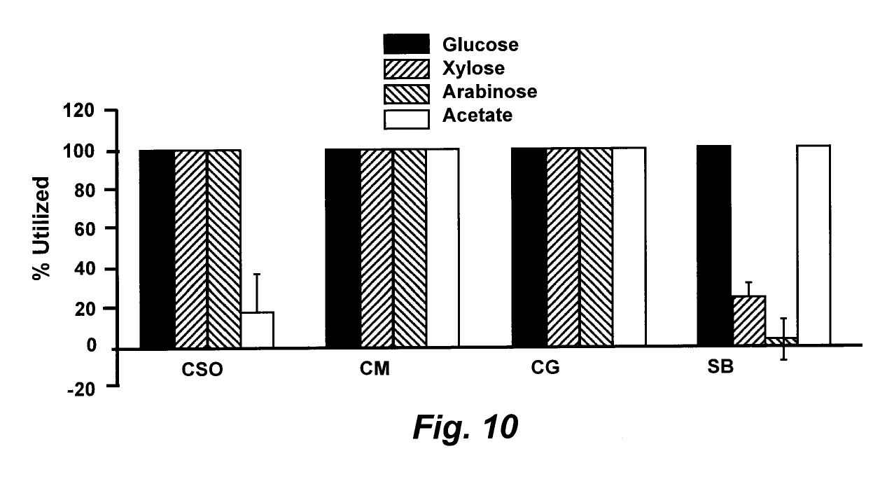

As shown in FIGS. 6-11, compared to all the strains tested, Chlamydomonas globosa showed a 466% increase in BG11 growth medium supplemented with 5% hydrolysates, and Chlorella minutissima showed 152%, 167%, and 126% increases in the BG11 medium supplemented with 5%, 10%, and 15% in chlorophyll a content, respectively.

In general, except for Chlamydomonas globosa, all the strains showed good biomass productivity on hydrolysate-containing media than the standard BG11. Chlorella minutissima grown in BG11 supplemented with 10% lignocellulosic hydrolysate recorded a 516% increase in biomass production than the control. The same culture recorded 291% and 220% increases in biomass production in BG11 supplemented with 15 and 5% hydrolysate, respectively and a 201% increase in deionized water supplemented with 10% hydrolysate.

Chlorella sorokiniana showed a 205% increase in the BG11 medium supplemented with 15% hydrolysate, whereas Scenedesmus bijuga showed 187 and 136% increases in BG11 growth medium supplemented with 10 and 15% hydrolysates, respectively.

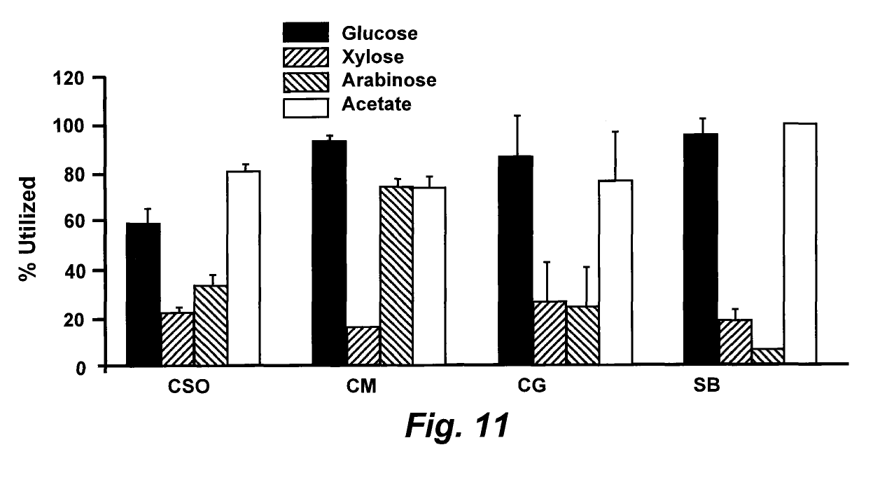

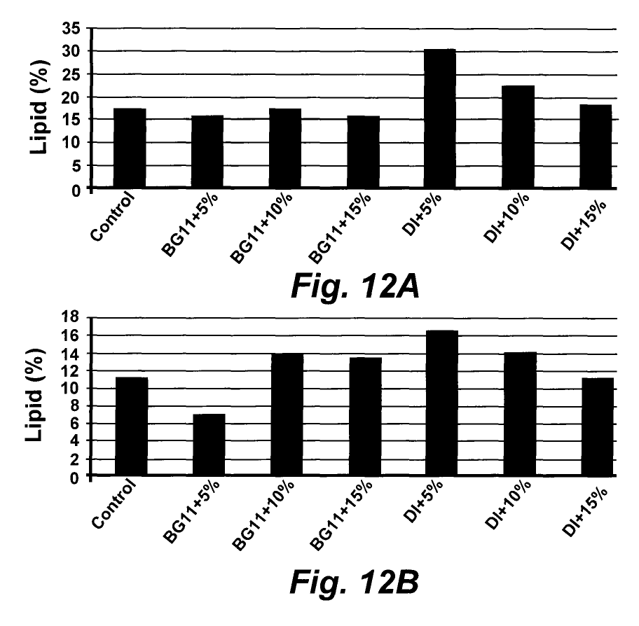

Chlorella sorokiniana recorded a 75% increase in lipid content, as shown in FIG. 12A, when grown in deionized water supplemented with 5% hydrolysates whereas DI water supplemented with 10% hydrolysates recorded only 28% increase when compared to the control (BG11 without hydrolysates).

Chlorella minutissima also showed highest increase in lipid content (50%) when grown in deionized water supplemented with 5% hydrolysates whereas DI water supplemented with 10% hydrolysates recorded only 28% increase when compared to the control (BG11 without hydrolysates). BG11 supplemented with 10% and 15% recorded 21-25% increase in lipids over control (FIG. 12B).

What is claimed:

1. A method of generating an algal biomass, comprising:
(a) forming an algal culture by combining:
(i) a population of algal cells characterized as proliferating in a culture medium comprising an industry wastewater; and
(ii) a culture medium comprising an industry wastewater, optionally, a municipal sewage effluent, and optionally a nutritional supplement, wherein said nutritional supplement increases the yield of algal culture compared to when the culture medium does not comprise the nutritional supplement, said nutritional supplement comprising an organic carbon source suitable for supporting the proliferation of a mixotrophic algal species, a mineral, a buffer, or a combination thereof; and
(b) maintaining the algal culture under conditions suitable for the proliferation of the population of algal cells, thereby forming an algal biomass.

2. The method of claim 1, wherein the industry wastewater is the effluent from an agricultural industry.

3. The method of claim 2, wherein the effluent is from a poultry industry, a non-poultry meat industry, or a plant-based industry.

4. The method of claim 1, wherein the industry wastewater is obtained from a non-agricultural industry.

5. The method of claim 1, wherein the nutritional supplement comprises at least one organic carbon source selected from the group consisting of: glucose, sucrose, arabinose, fructose, glycerol, methanol, acetate, a plant-based hydrolyzate, and any combination thereof.

6. The method according to claim 1, wherein the population of algal cells comprises a freshwater (non-marine) algal strain, a plurality of freshwater (non-marine) algal strains, a plurality of cyanobacter strains, a plurality of diatomaceous algal strains, or any combination thereof, and wherein at least one species of the population of algal cells is a mixotrophic alga.

7. The method of claim 1, wherein at least one algal strain of the population of algal cells is isolated from a source in contact with the wastewater effluent of an industry.

8. The method according to claim 1, wherein the population of algal cells comprises a strain of an algal genus selected from the group consisting of: Scenedesmus, Chlorella, Chlamydomonas, Scenedesmus and Chorella, Scenedesmus and Chlamydomonas, Chorella and Chlamydomonas, and Scenedesmus, Chorella, and Chlamydomonas.

9. The method of claim 1, wherein at least one algal strain of the population of algal cells is selected from the group consisting of: a Chlamydomonas sp., Chlorella vulgaris, Chlorella sorokiniana, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

10. The method of claim 1, wherein the population of algal cells is a consortium of algal cells comprising Chlamydomonas globosa, Chlorella minutissima, and Scenedesmus bijuga, and optionally Chlorella sorokiniana.

11. The method of claim 1, further comprising isolating the algal biomass from the medium.

12. A method of producing a biofuel from industrial wastewater comprising:
(a) forming an algal culture by combining:
(i) a population of algal cells characterized as proliferating in a culture medium comprising an industry wastewater; and
(ii) a culture medium comprising an industry wastewater, optionally, a municipal sewage effluent, and optionally a nutritional supplement, wherein said nutritional supplement increases the yield of algal culture compared to when the culture medium does not comprise the nutritional supplement, said nutritional supplement comprising an organic carbon source suitable for supporting the proliferation of a mixotrophic algal species, a mineral, a buffer, or a combination thereof, and wherein when the industry wastewater is an agricultural industry effluent, the agricultural industry is a poultry industry, a non-poultry meat industry, or a crop-based industry;
(b) maintaining the algal culture under conditions suitable for the proliferation of the population of algal cells, thereby forming an algal biomass;
(c) isolating the algal biomass from the medium; and
(d) obtaining from the isolated algal biomass a biofuel or a source of a biofuel,
wherein the step of obtaining from the isolated biomass a biofuel comprises the steps of isolating a lipid material from the biomass or converting the biomass to a biofuel, and wherein the isolated lipid material is converted to a biofuel.

13. The method of claim 12, wherein the nutritional supplement comprises at least one organic carbon source selected from the group consisting of: glucose, sucrose, arabinose, fructose, glycerol, methanol, acetate, a plant-based hydrolyzate, and any combination thereof.

14. The method according to claim 12, wherein the population of algal cells comprises a freshwater (non-marine) algal strain, a plurality of freshwater (non-marine) algal strains, a plurality of cyanobacter strains, a plurality of diatomaceous algal strains, or any combination thereof.

15. The method of claim 16, wherein at least one algal strain of the population of algal cells is isolated from a source in contact with the wastewater effluent of an industry.

16. The method according to claim 16, wherein the population of algal cells comprises an algal genus selected from the group consisting of: Scenedesmus, Chlorella, Chlamydomonas, Scenedesmus and Chorella, Scenedesmus and Chlamydomonas, Chorella and Chlamydomonas, and Scenedesmus, Chorella, and Chlamydomonas.

17. The method of claim 16, wherein at least one algal strain of the population of algal cells is selected from the group consisting of: a Chlamydomonas sp., Chlorella vulgaris, Chlorella sorokiniana, a Chlorococcaceae sp., Chlorococcum humicola, Coelastrum microporum, Gloeocystis vesiculosa, Monoraphidium mirabile, an Oedogonium sp., Oocystis lacustris, Scenedesmus abundans, Scenedesmus acuminatus, Scenedesmus acutus, Scenedesmus acutus alternans, Scenedesmus bicaudatus, Scenedesmus bijuga, Scenedesmus bijuga alternans, Scenedesmus denticulatus, Scenedesmus dimorphus, Scenedesmus incrassatulus, Scenedesmus obliquus, Scenedesmus quadricauda, Scenedesmus quadrispina, Scenedesmus serratus, a Stigeoclonium sp., Ulothrix variabilis, a Uroglena sp., an Anabaena sp, Aphanocapsa delicatissima, Aphanocapsa hyalina, an Aphanothece sp., Calothrix braunii, a Chroococcaceae sp., Chroococcus minutus, a Cylindrospermopsis sp., Leibleinia kryloviana, a Limnothrix sp., Limnothrix redekei, a Lyngbya sp., a Nostoc sp., an Oscillatoria sp., Oscillatoria tenuis, Planktolyngbya limnetica, Raphidiopsis curvata, Synechococcus elongatus, a Synechococcus sp., a Synechocystis sp., an Eunotia sp., Navicula pelliculosa, a Navicula sp., Nitzschia palea, Nitzschia amphibia, Nitzschia pura, Gomphonema parvulum, Gomphonema gracile, and a Rhodomonas sp.

18. The method of claim 16, wherein the population of algal cells is a consortium of algal cells comprising Chlamydomonas globosa, Chlorella minutissima, and Scenedesmus bijuga, and optionally Chlorella sorokiniana.
